(19) United States
(12) Patent Application Publication
(10) Pub. No.: US 2008/0113865 A1
(43) Pub. Date: May 15, 2008

(54) GLEDITSIA AMORPHOIDES SEEDLESS POD EXTRACT AND ITS USE AS AN AGRICULTURAL ADJUVANT

(75) Inventor: Gustavo Prola, Ciudad de Buenos Aires (AR)

Correspondence Address: HUGHES HUBBARD & REED LLP, ONE BATTERY PARK PLAZA, NEW YORK, NY, 10004, US

(73) Assignee: IDEASUPPLY.COM ARGENTINA S.A., Ciudad de Buenos Aires (AR)

(21) Appl. No.: 11/616,975

(22) Filed: Dec. 28, 2006

(30) Foreign Application Priority Data
Dec. 28, 2005 (AR) P050105589

(51) Int. Cl. A01N 43/40; A01N 25/00; A61K 47/46; A01P 7/04; A01P 13/00; A01P 3/00

(52) U.S. Cl. 504/250; 504/358; 514/783

(57) ABSTRACT

Extract of the seedless pod of Gleditsia Amorphoides and its use as adjuvant in agriculture, which has a content of soluble solids between 250-270 g/kg; triterpene saponins between 60-75 g/kg; a conductivity (5° Brix) of 6+/−4 mS/cm; an absorbance of 400 nm (at 1.1% of the product in water)<0.500 UA; a foam (5° Brix) of 160 ml; a (direct) pH equal to 3.9+/−0.3; while its chemical assessment in g. per 100 g. of the extract expressed as elements amounts to a total Ni=0.51; assimilable P=0.28; K=1.22; S=0.63; Mg=0.08 and Fe=0.0022; while assimilable P (expressed as 05P)=0.64 and K (expressed as KO)=1.47; likewise, the total amount of phenols is approximately 8.5 and 10%, and that of tannins is between 0.9 and 1.5%.

This application claims benefit of Argentinean Application No. P050105589, filed on Dec. 28, 2005, which is hereby incorporated by reference.

### BACKGROUND OF THE INVENTION

1. Field of the Invention

This invention is related to agricultural products in general, and, in particular, to an organic agricultural adjuvant obtained from the treatment given to the fruit—consisting of seedless pods—of a tree popularly known as “Espina de Corona or Coronillo,” whose scientific name is Gleditsia Amorphoides.

2. Description of Prior Art

Experts know that the Argentine agricultural market uses to a large extent the so-called surfactant agents, products that reduce water surface tension, thus favoring the penetrability of the various agrochemical compounds used in all field and/or intensive crops throughout the country.

Eighty percent of these surfactants are used in different herbicide, insecticide and fungicide applications, some of which are already formulated with surfactants, the remaining twenty percent corresponding to organochlorinated compounds, widely used nowadays. All the above are chemicals, i.e., non-organic products.

A solution containing the active product, biocide or fertilizer, surfactant and water should be prepared for spraying.

It is common knowledge that surfactant or co-adjuvant products are classified into:
a) Non-ionic: based on ethoxylated nonylphenol-ethoxylated alcohol
b) Anionic: Sodium dodecyl benzene sulfonate-sodium lauryl ethoxy sulfate-calcium lignosulphonate.
c) Cationic: Benzalkonium chloride-ethoxylated amines.
d) Amphoteric: Alkylbetaine-sulfobetaine.
e) Siliconated: Organo-siliconated, they basically feature the same structure as conventional surfactants, though their lipophilic portion is made up of xiloxanes, which have a Si group in their structure, in addition to the oxyethylene hydrophylite chain.
f) Oils: Compounds derived from petroleum and vegetable oils; they do not have a significant hypo-tightening activity.

As mentioned above, most of these products (80%) belong to the ionic group, i.e., they derive from chemical syntheses which are being forbidden in the European Economic Community due to their proven carcinogenic and teratogenic effects, and whose reduced use in the agricultural field evidences its trend to be phased out and replaced by natural, non-pollutant products.

Most commercial glyphosates (isopropylamine salt) have low toxicity; however, when adding the surfactant required to fulfill they function as powerful herbicides, e.g., POEA (poly-oxy-ethylene-amine), their toxicity increases from 3 to 5-fold.

So, upon mixing, the commercial formulation product toxicity is leveraged, featuring toxicological characteristics absent in glyphosate alone, resulting in a pesticide which causes severe poisoning, gastrointestinal, pulmonary, respiratory, and kidney problems, as well as central nervous system disorders to those who handle it. It causes blood pressure to increase, destroys red cells, and causes skin and eye irritation, either accidental or due to handling and use in the field, in addition to severe pollution of the land, with significant damage to the natural habitat as it has an impact on all human beings or animals.

It should be noted that, when applied to fertilizers, several formulations are used in the prior art. They are listed below by their trade name:

SOL UAN, formulated from a basis of urea and liquid ammonium nitrate, contains a 32% Ni.

SOL PLUS, 12% Ni and 26% S, a mixture of both with variable doses of Ni and S, compatible with any of the conventional herbicides, is also used.

The product marketed as SOL FOS contains 9% Ni and 27% Ph. There is another product called “FOLIAR SOL U,” with 20% Ni.

The so-called UAN 32 is formulated with Ni from three different sources (urea, ammoniac and nitric sources.)

AMMONIUM SULFATE is a widely used compound produced with Ni from nitric and ammoniac sources, S, and their mixtures.

Among conventional liquid fertilizers, we can mention WUXAL Ca (10% Ni+Ca), NITROPLUS 9 and 18 (10% Ni+Ca), CITROLINO (10% Ni+trace elements), and NITROSKA FOLIAR (10% Ni+4% Ph+7% K.)

These conventional fertilizers do not interfere with the one referred to in these specifications, based on the discovery of an adjuvant or humectant from a natural resource present in the seedless fruit of Espina de Corona, scientifically known as Gleditsia Amorphoides, combined with water-dissolved granular Urea. Thus, the result is a nitrogenous fertilizer with the unique characteristics of a built-in organic humectant in its formulation.

The search for Numbers 05/10/118-119 and 120 conducted by INPI's Technological Information Department in its databases evidenced no reference to the production of an adjuvant from the above tree species.

For illustration purposes only, the Department added some references to patents which do not interfere with this invention, though they deal with pesticides and germicides derived from vegetable products. See the list below:

CN1102056 “Vegetable germicide,” belonging to Sanlian Biochemistry Factory Y (CN), 1995. This invention describes a plant germicide. It consists of an effective combination of tricodermus fungus and traditional Chinese medicine consisting of philodendron bark, asteraceae, rhubarb and Gleditsia Sinensis in a suitable proportion used to control downy mildew or rust in vegetables and fruit trees.

CN1122653 “Emulsion of Yansensu and preparation method thereof,” belonging to Bingyun Yang (CN); Zihe Sui (CN); Ren Jiuwen (CN), 1996. The pesticide emulsion contains 40-50 extract; 18-25% ethylic alcohol, and 30-40% adjuvant, where the extract consists of (weight %) 50-60% tobacco leaves; 15-20% flavescent sophora root; 15-20% stemona root; 5-10% Gleditsia fruit. The preparation method includes cutting, washing, soaking, boiling for 100-120 minutes, precipitating, centrifugation filtering, concentrating, adding ethylic alcohol, adding an emulsifier and synergistic penetrant. It is a highly effective, broad-spectrum, slightly toxic product, but harmless to human beings and animals.

CN1153599 “Vegetable pesticide,” belonging to Xu Guizhu (CN); Fenglan (CN); Xu qinghui (CN), 1997. Pesticide for vegetables prepared with natural vegetable materials, including semen strychni, croton, almond, Gleditsia Sinensis, white pepper, malt, etc., through soaking in a liquid mixture of volatile solvents—methanol, ethylic alcohol and chloroform—to extract its active components, which are then mixed. It provides strong pest destruction effects on plants and crops, and can leverage resistance to crop and plant diseases with no harmful effect on human beings or farm animals.

CN1168228 “Wettable powder pesticide and preparation thereof,” belonging to Liu Lidong (CN); Li Jinqing (CN), 1997. It consists of bavistin, thiram, penetrant, Gleditsia Sinensis powder, and clay powder. It provides a strong systemic action and a fungicide effect for fungal diseases in fruit trees and can be used together with organophosphoric pesticides and piretrine. It is mostly used to prevent and control pear black core fungus. Its production process is simple, with zero water, gas, waste, or environmental pollution.

CN1433696 Wnag Huaiyong (CN): zhang xiating (CN), 2003. It consists of a dry pollen agent and a pyrethrin wettable powder agent.” It refers to a type of pyrethrum desiccated pollen agent, its wettable powders, and its preparation method, which includes the following steps: spraying pyrethrum stems, leaves, and dry flowers adding diatom powder, talc or clay at a 10-30%/weight of pyrethrum stems and leaves, thoroughly mixed, sprayed, screened with a mesh 200 to get the pyrethrum. Based on the percentage over weight of the above pyrethrum powder, 3-5% TBHQ or BHT, 5-15% Gleditsia fruit or saponified tea, 1-3% synergistic ether or octachlorine-dipropylic ether or synergistic amine are mixed, sprayed, and screened with a 200 mesh.

The above pyrethrum pollen and all ancillary materials are thoroughly mixed to get the invented wettable powder.

It should be noted once again that INPI's report, after searching its national and international databases, evidences no reference regarding the development of an adjuvant compound from Gleditsia Amorphoides as stated in this document.

### BRIEF SUMMARY OF THE INVENTION

This invention is related to an organic agricultural adjuvant obtained from the treatment given to the fruit—consisting of seedless pods—of a tree popularly known as “Espina de Corona” or “Coronillo”, whose scientific name is Gleditsia Amorphoides.

An additional purpose of this invention is to provide an adjuvant that could be used to carry out any kind of terrestrial and aerial sprayings (combined with other agrochemical products, such as herbicides, insecticides, fungicides and biocides), or for the commercial formulation of glyphosate (48% isopropylamine salt) or other agrochemical formulations.

This invention consists mainly in taking out from the above tree seedless pod, by means of a specific industrial process, an organic extract consisting of an adjuvant that, when added to or combined with other active products, contributes to protecting the environment and reducing the environmental impact that most adjuvants, humectants, or surfactant agents have when combined with other commercial agrochemicals.

Either in its liquid or solid states, the main advantages of this invention in terms of its physical-chemical features and based on a real case where it was used in glyphosate formulation, are as follows: lower cost; the elimination of antifoaming agents due to its low-foam action; the need for lower doses than those of the current formulated product, from 10 to 1 of the surfactant agent used, thus reducing handling and warehouse costs; and, essentially, reducing glyphosate toxicity risks.

Based on its distinctive features as compared to other adjuvant agents used for crop spraying, we can point out that:

It is an organic product and only reduced doses, 4 to 1 against commercial products, are required, thus resulting in handling and warehouse cost reductions, among others. Its price is cheaper, it reduces final product toxicity risks, and it is not harmful to the environment, human beings, or animals.

In general, references are made to the step after taking the aqueous extract from Gleditsia Amorphoides. Its use, for instance, in a solid state shows no evident problem.

In order to properly specify the data leading the appellant to discover the co-adjuvant described herein, it seems reasonable to provide a brief overview of the above mentioned plant.

The Gleditsia Amorphoides tree, commonly known as Espina Corona, reaches a maximum height of 15 m and a trunk diameter of 30-60 cm, which, just like its branches, is full of ramified large reddish thorns. Its fruit is a legume, generally hooked-shaped, rigid, blackish, indehiscent and somewhat pulpy with a slightly septate endocarp containing from 6 to 10 seeds.

This species is extremely important in the agricultural exploitation and logging in the Northern Argentina. In addition to the importance of its wood for carpentry, it is characterized by the production of a very good hydrocolloid thickener from its seeds, which is fit as gelling agent for food products such as marmalades, jellies, and solid jams, and as emulsifier for mayonnaise and sauces as a substitute for fat.

The wastes of the manufacturing process of the above seed thickener consist of the pod fragmented remains, which are set aside in the facility sieves.

The applicant decided to conduct some research on the remaining bagasse, a product till now is disposable, by subjecting it to another industrial process, which resulted, through dissolution, in the active product contained in the bagasse. Moreover, as proved by the applicant with the experiments conducted, the resulting product turned out to be a natural co-adjuvant, as it consists of a significant amount of triterpene saponins.

To make this invention understandable so that it can be easily put into practice, the following paragraphs will provide an accurate technical and legal description of some forms of execution, including references to the attached figures, being them for illustration purposes but not limited to the uses of the invention, whose components could be selected from different equivalents, with no subsequent deviation from the invention principles established in this documentation.

### BRIEF DESCRIPTION OF THE DRAWINGS

FIG. 1 shows grass control percentages.

FIG. 2 shows dicotyledonae control percentages.

FIG. 3 shows overall weed control percentages.

### DETAILED DESCRIPTION OF THE INVENTION

Pursuant to the above immediate assays conducted, the co-adjuvant product of this invention is an aqueous product rich in the saponin portion of Gleditsia Amorphoides seedless fruit, such portion provides it with surfactant characteristics which make it suitable for use in herbicides, fertilizers, and other agrochemicals.

It is common knowledge that saponins are molecules whose structure has triterpene cores linked to glycosides which add them a hydrophilic-lipophilic nature that favors the penetration of the active principle and its flow along the plant vascular system. Therefore, co-adjuvant products added to formulations, e.g., herbicides, also reduce surface tension of the spraying mixtures, thus increasing the contact surface and the adherence of the herbicide active product, in this case, and the application surface.

Gleditsia Amorphoides co-adjuvant composition:

Content of soluble solids=250-270 g/kg

Triterpene saponins=60-75 g/kg

Conductivity (5° Brix)=6+/−4 mS/cm

Absorbance at 400 nm (at 1.1% of the product in water=<0.500 UA

Foam (% Brix)=160 ml

pH (direct)=3.9+/−0.3

Solubility=fully water-soluble.

Recommended dosage: direct addition to a spraying solution=from 30 to 60 ml per 100 liters of solution.

For herbicide formulations=from 1.0 to 1.5% v/v of product in final product formulation.

Compatible preservative=1 g/l of Na benzoate.

Product shelf life=2 years at 5-25° C. in closed container.

The analysis conducted at Microquim SA laboratories of a sample of Gleditsia Amorphoides aqueous extract obtained based on the invention resulted in a titration of gram/100 g of sample of:

Total Ni=0.51; assimilable P (expressed as element)=0.28 assimilable P (expressed as 05P)=0.64; K (expressed as element)=1.22; K (expressed as KO)=1.47; S (expressed as element)=0.63; Mg (expressed as element)=0.08; Co and Mo (expressed as elements)=undetectable; Fe (expressed as element)=0.0022.

The above titration was conducted based on the following methodology:

Total Ni=Kjeldahl's Method; assimilable P=A.O.A.C. 993.31; K, Mg, Co, Mo, and Fe=atomic absorption spectrophotometry; and for S=gravimetry as per A.O.A.C.

This study is supplemented with others which resulted in a total amount of phenols=between 8.5 and 10%, approximately, measured with Folin-Cicaltea's Method.

Tannins=0.9-1.5% measured by Makkar H. P. S. Bluemmel. M. Borowy N. K (J. Sci. Food Agric. 61 1993 161-165.

Saponins=20-25%, approximately. Estimated value by weight separation with tangential filtration.

Another use of the invention deals with the possibility of a fully organic fertilizer, especially fit to make nutritional adjustments in either intensive or field crops, fruit, vegetable, and forest crops, among others, in order to improve their yield. Moreover, it is particularly effective when a quick response is needed.

This is another advantage of the purpose of this invention, as the organic co-adjuvant produced and added to a fertilizer formulation has a strong surfactant effect on waxy vegetable strata, which favors the active product quick absorption and transport.

When added to the fertilizer, it helps this organic molecule, obtained from concentrated vegetable extracts, act in association and synergically with conventional agrochemicals, being compatible with most of them.

The invention obtained from processing Gleditsia Amorphoides seedless fruit essentially results in an important use and provides the advantage of allowing for the industrial production of a fully organic nitrogenous foliar liquid fertilizer, thus avoiding the use of non-environmental-friendly products.

Moreover, the fertilizer which includes the addition of the organic humectant extracted from Gleditsia Amorphoides fruit is unique, i.e., it is a nitrogenous liquid fertilizer plus an organic humectant, i.e., the adjuvant.

Therefore, the invention adjuvant or humectant can be added to a fertilizer that will consist of the aqueous extract resulting from processing Gleditsia Amorphoide fruits, which is used as the first component of the formulation of a liquid fertilizer that has a nitrogenous second component.

The second component is a nitrogenous fertilizer consisting of 20% Ni, which can be obtained from the dissolution in water of granular urea with low biuret content—less than 0.5%. The neutral pH water used shows no hardness (Ca+, Mg+.)

Then, the formulation is prepared by mixing one liter of the solution of granular urea in water with a 20% Ni—the second component—with 60 cc of the co-adjuvant—the first product component.

To supplement the above, find below the analysis of a nitrogenous fertilizer containing the invention adjuvant, conducted by SENASA, Assay Report LF 9850, dated Dec. 19, 2005. The results are as follows:

Organic Ni=from 10 to 46 g/10 g

Surface tension=36.6 dynes/cm

pH=6.9

Biuret=0.15 g/100 g

Where the Ni was assessed with Kjeldhal's Method using semiautomatic equipment (PCP 1-FFC LF No. 003).

Surface tension measured with DuNouy's tensiometer. (PCP 1-FF LF No. 017.)

pH assessment (PCP 1-FFC LF No. 018.)

Biuret assessment using UV-Vis spectrometry (PCP 1-FFC LC No. 020.)

Foliar Fertilizers Resolution SENASA (National Agri-food Quality and Health Service) No. 53/76.

In connection with co-adjuvant toxicity, it should be noted that several assays are currently being conducted at Microquim Laboratories, out of which we can provide an overview of those reported to date, included in the following summary:

CU (so called for simplicity purposes) Surfactant Product Data Summary, belonging to the company: Ideasupply.com S.A:

Oral Toxicity in rats: >3,000 mg/Kg—Usually non-hazardous products. Dermal toxicity in rats: >4,000 mg/Kg—Usually non-hazardous products.

Inhalation toxicity: 4.89 mg/l—(maximum nebulizable concentration.)

Primary Dermal Irritation: Non-irritant Product.

Eye Irritation: Ongoing.

Sensitization Index: Ongoing.

Ames Test: Cytotoxicity: Non-toxic Product.

Mutagenicity: Ongoing.

Another application with agrochemicals was conducted by assessing the influence of the “CU” co-adjuvant of the invention on the activity of defoliant “Paraquat” in alfalfa (Medicago Sativa L.)

For such purpose, the effect of the addition of the surfactant Coronillo Ultra (CU) at 0.25% v/v and emulsion oil at 1% v/v to defoliant Paraquat at a 1 l/ha concentration, in conditions of high and low temperatures during both the day and at night on alfalfa was assessed.

Five days after the treatment, it was observed that, at day-night temperatures between 30/21° C., defoliation amounted to 17% with no addition of adjuvants, 37% with the addition of oil, 68% with CU, and 80% with both co-adjuvants; whereas at temperatures between 21/123° C., defoliation was 10% lower for all treatments.

After 10 days, there were no differences among treatments at high or low temperatures.

The aftergrowth in both environmental conditions decreased by 75% with the addition of CU, and 86% with CU plus oil in mechanical vis-à-vis manual defoliation.

The absorption of the active is impacted by temperatures during application. The use of CU plus shaking favored the effect of the desiccant product.

Efficacy of “avemectin” and the CU co-adjuvant of the invention to control leafmining moth (Phyllocnistis Citrella Stainton.)

Two assays were designed in order to assess the effect of two concentrations of the Coronillo Ultra surfactant and combinations of both products to control leafmining moth in citrus (Phyllocnistis Citrella Stainton.)

One of the assays was geared to testing the action of the direct contact of the products, both pure and in mixtures, with the larvae of the second and third stages by injecting the solution into the tunnels. The second assay aimed at assessing the effect of its application on the foliar surface affected with tunnels. In both assays, methylene blue dye was used as tracer to assess the flow of the solution inside the tunnel and the larvae.

In the treatments applied over the foliar surface, effects were noticed only when the active penetrated the epidermis and came into contact with the insect inside the tunnel.

The CU co-adjuvant increased the transfer of solution across the tunnels and controlled larvae with the injection of a 0.5% concentration.

High concentrations of avemectin proved effective when applied over the foliar surface, while low doses had the same effect when injected.

The mixtures of the CU co-adjuvant 0.25% with avemectin resulted in increased pest control against the different doses of the active without co-adjuvant. The leafmining moth was 90% controlled when using 0.25% plus 0.15% avemectin. Control shows a positive correlation in assayed treatments combining this co-adjuvant with avemectin.

These results evidence a reduction in the concentration of avemectin against the increased use of co-adjuvant to achieve similar control over Phyllocnisis spp.

Finally, the above treatments were tested on the field, in a five-year vegetable garden. The doses of avecmetin mixed with the co-adjuvant resulted in a 75% control of the larvae and pupas against the full control sample.

Treatments with no surfactant showed irrelevant and heterogeneous results.

Treatments with avemectin evidenced a low control percentage after 14 days. However, treatments with +0.25% avecmetin and +0.5 co-adjuvant showed significant control between days 21 and 28 post-application, reducing larvae by 85%.

To provide another application example, a field trial of glyphosate formulated with 1% of the concentrated surfactant obtained from the tree Coronillo (which we continue referring to as “CU”) as per the invention was conducted to assess grass and broad-leafed weed control.

Several research studies point out the efficacy of the glyphosate herbicide at 960 g.i.a/ha to control weed in corn and soy pre-planting on the Pampas plain with direct tilling systems and applied to weed foliage and as a pre-emergence herbicide. However, within the current targeted sustainable agriculture model, it is not only necessary to reduce soil erosion, but there should be a reasonable use of pesticides as well. The use of co-adjuvants may provide for an easy reduction of herbicide doses by increasing their efficacy, which is convenient from both the environmental and economic perspectives.

On the other hand, as mentioned above, co-adjuvants proved to increase glyphosate phytotoxicity. However, variations among the effects of the different assessed surfactants have been found. Their use improves soaking and increases herbicide retention and penetration through two possible action mechanisms: expansion of the cuticle polymer matrix or solubility.

Nevertheless, as not all co-adjuvant action mechanisms are fully understood, it is necessary to conduct factorial trials on herbicides together with co-adjuvants in order to learn surfactant synergic or conflicting effects on herbicide efficacy.

The goal was to assess the co-adjuvant effect on the activity of the glyphosate herbicide used to control grass and broad-leafed weed in summer crop pre-planting.

The assay was conducted at Establecimiento San Carlos, located in Ingeniero Silveyra, District of Chacabuco, Province of Buenos Aires. The area is characterized by a 900/1,200 mm/year rainfall pattern, with bimodal distribution, and temperate winters with a relative humidity over 60%. Potential evapotranspiration amounts to 1,450 mm/year. Soils as classified as silty sandy clay loam, deep, and with a permeable B horizon, 2.5/2.8% of organic matter, and a 5.8-6.3 pH.

The used statistical design was a randomized block design with five repetitions. A total of 7 treatments were assessed: glyphosate 48% at a 672 g.i.a./ha dose, solution and mixed with the invention “CU” surfactant at 0.0; 0.02; 0.03; 0.04; 0.05; and 0.06% v/v doses. A full control sample treatment was included. Overall, results showed significant differences for the study variables: Surfactant effect and weed control percentages.

A preliminary study was conducted to survey the weed population in the assay area, where the dominant grass-type species in the autumn, winter, and spring seasons are as follows: rescue grass (bromus unioloides), large crabgrass (Digitaria sanguinalis), bermuda grass (Cynodon dactilon), giant foxtail (Setaria spp), annual ryegrass (Lolium multiflorum), etc.; dicotyledonae: black bindweed (Polygonum convolvulus), wild buckwheat (Poligomun aviculare), stinging nettle (Urtica Ureas), wedelia (Wedelia glauca), crested anoda (Anoda cristata), lamb's-quarters (Chenopodium album), and cultivated radish (Raphanus sativus.)

They were evenly distributed and in full development (in general, between 3 and 9 pairs of leaflets) with no stress signs.

Treatments were applied on weed post-emergence. A back-sprayer with a constant pressure electromechanical pump was used at a 4 kg working pressure and a 116 l/ha field effective flow.

Six treatments plus a control sample with five repetitions were assessed in a randomized block design.

The experimental unit consisted of a 30 sq. m (3×10) plot. The 1.5 m central band was considered for the assessment.

TABLE NO. 1
Treatments

| Treatment | Description |
|---|---|
| 1 | Glyphosate 672 g.i.a./ha + CU 0.06% v/v |
| 2 | Glyphosate 672 g.i.a./ha + CU 0.05% v/v |
| 3 | Glyphosate 672 g.i.a./ha + CU 0.04% v/v |
| 4 | Glyphosate 672 g.i.a./ha + CU 0.03% v/v |
| 5 | Glyphosate 672 g.i.a./ha + CU 0.02% v/v |
| 6 | Glyphosate 672 g.i.a./ha |
| 7 | Full control sample. Water + CU 0.06% v/v |

Treatments were assessed in terms of the grass and broad-leaf weed control percentage and the quantitative method full control sample on days 14/21 after the application based on the following equation:

$$\% control = \frac{\begin{matrix}( control sample weed weight - \\ treatment weed weight ) \times 100\end{matrix}}{control sample weed weight}$$

Results were compared to the scale approved by Asociación Latinoamericana de Malezas (ALAM), (Latin American Weed Association).

For the statistical analysis, results were loaded to the SAS (Statistical Analysis System) package, and mean separation tests for significant effects were run by Tukey.

Results and Analysis:

Grass weed control percentage. The variance analysis for grass control percentage evidenced highly significant differences (P<0.01) among the assessed treatments. Table 3 shows the mean test for this variable. The highest control percentage was achieved with treatment No. 1 (Gl+CU 0.06% v/v), value rated as very good (Table 1), there being no statistical difference between this and treatments 2 (Gl+CU 0.05) and 3 (Gl+CU 0.04), rated as good, and treatment 4 (Gl+CU 0.03% v/v), rated as satisfactory.

However, statistical differences were evidenced in treatments 5 (Gl+CU 0.02% v/v) and 6 (Gl), which showed 58.9% and 40.5% controls, respectively, and were rated as fair, thus providing no satisfactory grass weed control. The above results suggest that the combination of CU plus glyphosate at low co-adjuvant concentrations with the selected herbicide concentration failed to provide a suitable and satisfactory control over the study grass weeds, such as Cynodon and Digitaria.

TABLE NO. 2
Weed control degree as per ALAM

| Rate (%) | Control Degree |
|---|---|
| 0-40 | None or poor |
| 41-60 | Fair |
| 61-70 | Satisfactory |
| 71-80 | Good |
| 81-90 | Very good |
| 91-100 | Excellent |

TABLE NO. 3
Grass weed control percentage

| Treatment | Description | Control % |
|---|---|---|
| 1 | Glr 672 g.i.a./ha + CU 0.06% v/v | 84.3^{a} - very good |
| 2 | Glr 672 g.i.a./ha + CU 0.05% v/v | 76.7^{ab} - good |
| 3 | Glr 672 g.i.a./ha + CU 0.04% v/v | 71.3^{ab} - good |
| 4 | Glr 672 g.i.a./ha + CU 0.03% v/v | 70.6^{ab} - satisfactory |
| 5 | Glr 672 g.i.a./ha + CU 0.02% v/v | 58.0^{bc} - fair |
| 6 | Glr 672 g.i.a./ha | 40.5^{c} - fair |

Glr commercial glyphosate a, b, c: Treatments with the same letters show no significant differences. Mean test ran by Tukey (5%.)

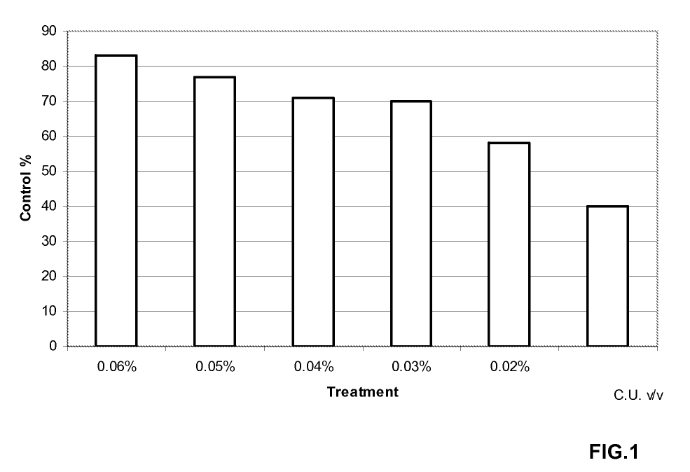

See FIG. 1.

Broad-leafed weed control percentage. Highly significant differences (P<0.01) among treatments were observed. The mean test (Table No. 4) evidences treatment 2 (Gl+CU 0.05% v/v) with a very good control percentage (80.9%). However, there is no statistical difference between this and treatments 1 (CU 0.06% v/v), 3 (CU 0.04% v/v) and 4 (CU 0.03% v/v), though that is not the case with treatment 5 (CU 0.02% v/v.) As per the ALAM scale, all treatments where the invention CU surfactant was mixed with Glr glyphosate ranked at a good, tending to very good, control level.

This leads us to think that the co-adjuvant has a synergic effect on glyphosate, thus increasing its efficacy in broad-leafed weed control.

Moreover, it was observed that, when working alone (treatment 6), glyphosate showed the lowest efficacy in the control of this type of weed, especially regarding Convolvulus spp and Wedella spp., prevailing in the assay area.

TABLE NO. 4
Broad-leafed weed control percentage

| Treatment | Description | Control % |
|---|---|---|
| 1 | Glr 672 g.i.a./ha + CU 0.06% v/v | 90.4^{a} - very good |
| 2 | Glr 672 g.i.a./ha + CU 0.05% v/v | 80.9^{a} - very good |
| 3 | Glr 672 g.i.a./ha + CU 0.04% v/v | 79.1^{abc} - good |
| 4 | Glr 672 g.i.a./ha + CU 0.03% v/v | 78.4^{abc} - good |
| 5 | Glr 672 g.i.a./ha + CU 0.02% v/v | 65.6^{abc} - satisfactory |
| 6 | Glr 672 g.i.a./ha | 61.4^{c} - satisfactory |

Glr commercial glyphosate a, b, c: Treatments with the same letter show no significant differences. Mean test ran by Tukey (5%.)

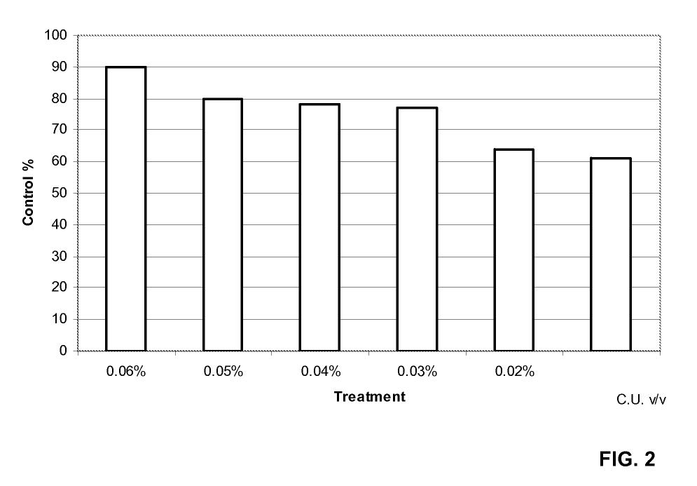

See FIG. 2.

Total weed control percentage. The statistical analysis showed highly significant differences among the assessed treatments. Table No. 5 shows that the best control was achieved with treatment 1 (CU 0.06% v/v), with an 81.3 control percentage rated as very good. There are no statistical differences between treatment 1 (CU 0.06% v/v) and treatments 2 (CU 0.05% v/v), 3 (CU 0.04% v/v) and 4 (CU 0.03% v/v), all rated as good. However, there is no significant difference between treatment 2 (CU 0.05% v/v) and treatments 5 (CU 0.02% v/v) and 6 (Gl), which evidenced only a 59.8 and 46.9 total weed control percentage, respectively, and rated just as satisfactory by ALAM.

TABLE NO. 5
Total weed control percentage

| Treatment | Description | Control % |
|---|---|---|
| 1 | Glr 672 g.i.a./ha + CU 0.06% v/v | 81.3^{a} - very good |
| 2 | Glr 672 g.i.a./ha + CU 0.05% v/v | 73.6^{ab} - good |
| 3 | Glr 672 g.i.a./ha + CU 0.04% v/v | 73.6^{ab} - good |
| 4 | Glr 672 g.i.a./ha + CU 0.03% v/v | 72.1^{ab} - good |
| 5 | Glr 672 g.i.a./ha + CU 0.02% v/v | 59.8^{bc} - fair |
| 6 | Glr 672 g.i.a./ha | 46.9^{c} - fair |

Glr commercial glyphosate a, b, c: Treatments with the same letter show no significant differences. Mean test ran by Tukey (5%.)

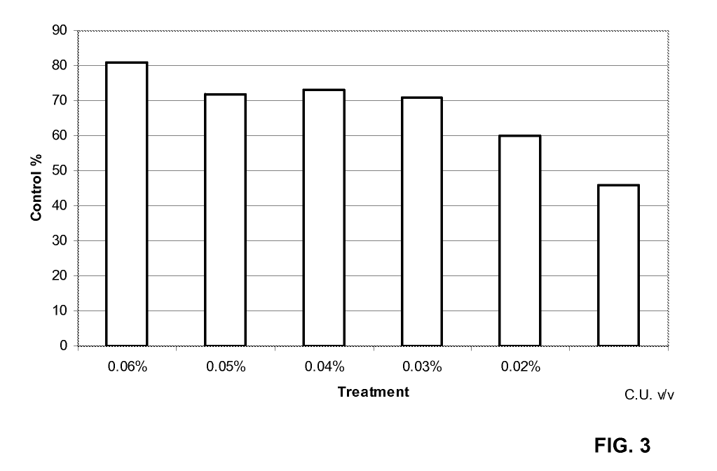

See FIG. 3.

### CONCLUSIONS

The use of the invention agricultural CU co-adjuvant at concentrations of 0.03; 0.04; 0.05 and 0.06% v/v significantly increases the activity of the commercial glyphosate herbicide in terms of the control of weeds present in the assay area against the control sample with no surfactant.

For some weeds, concentrations of 0.06% v/v reduce the control over both biochemical and physical factors, such as broth run-off through the leaf mass.

Likewise, we conducted an efficacy assay on grass and broad-leafed weed control using glyphosate formulated with the invention CU co-adjuvant concentrate 1%, whose protocols prove the advantages it offers and are attached as “Reference A” to this document.

As mentioned above, the use of surfactants improves both distribution and its subsequent retention on the foliar surface, or translocation, thus reducing both dilution and the effect of the physical force of rain on the active removal.

It is interesting to learn the effect of the invention surfactant on the glyphosate herbicide retention and activity when exposed to rain after treatment.

For such purpose, we assessed the influence of three concentrations of the aqueous extract consisting of the invention co-adjuvant, whose comprehensive analysis is attached to this document as “Reference B”. We analyzed the behavior of several surfactants frequently used in agriculture in different situations and at different intervals. The results evidence the invention co-adjuvant optimal behavior.

Also, find attached as “Reference C” a supplementary field trial of glyphosate formulated with the surfactant extract. The characteristic of this trial is that the invention concentrate is 1% over grass and broad-leafed weed control, and was conducted under stringent analysis conditions for different formulations of the active product, similar to the one described above, which provided optimal results.

Therefore, we have included some of the possible uses of the adjuvant in agrochemicals, which realize the invention and its action. It was noted that several different secondary active components can be applied to each specific formulation, preferably obtained from natural elements to keep the organic nature of the product to be used in agriculture, active products already known, associated, depending on the invention, with the first essential component made up of the natural co-adjuvant resulting from processing Gleditsia Amorphoide seedless fruit in a chemically pure aqueous solution.

I claim:

1. Extract of the seedless pod of Gleditsia Amorphoides and its use as adjuvant in agriculture, which has a content of soluble solids between 250-270 g/kg; triterpene saponins between 60-75 g/kg; a conductivity (5° Brix) of 6+/−4 mS/cm; an absorbance of 400 nm (at 1.1% of the product in water)<0.500 UA; a foam (5° Brix) of 160 ml; a (direct) pH equal to 3.9+/−0.3; while its chemical assessment in g. per 100 g. of the extract expressed as elements amounts to a total Ni=0.51; assimilable P=0.28; K=1.22; S=0.63; Mg=0.08 and Fe=0.0022; while assimilable P (expressed as 05P)=0.64 and K (expressed as KO)=1.47; likewise, the total amount of phenols is approximately 8.5 and 10%, and that of tannins is between 0.9 and 1.5%.

2. Extract of the seedless pod of Gleditsia Amorphoides and its use as adjuvant in agriculture, as per claim 1, wherein said adjuvant extract has undergone assays as follows: Oral toxicity in rats >4,000 mg/kg; inhalation toxicity at maximum nebulizable concentration of 4.89 mg/l; primary dermal irritation and Ames Test, cytotoxicity: they all show that it lacks toxicity.

3. Extract of the seedless pod of Gleditsia Amorphoides and its use as adjuvant in agriculture, as per claim 1, wherein its absorbance at 3° Brix is of 1,230 and 1,120.

4. Extract of the seedless pod of Gleditsia Amorphoides and its use as adjuvant in agriculture, as per claim 1, wherein said extract is highly soluble in water.

5. Extract of the seedless pod of Gleditsia Amorphoides and its use as adjuvant in agriculture, as per claim 1, wherein, as a compatible preservative, 1 g/l of Na benzoate is used.

6. Extract of the seedless pod of Gleditsia Amorphoides and its use as adjuvant in agriculture, as per claim 1, wherein said extract is obtained as a result of the treatment of the bagasse composed of the seedless pod of Gleditsia Amorphoides.

7. Use as adjuvant in agriculture of the extract of the seedless pod of Gleditsia Amorphoides, as per claim 1, wherein its use as a fertilizer is comprised of two essential components, the first of which consists of the organic natural co-adjuvant extract composed of a 30/60 cc aqueous solution of said extract of the fruit of Gleditsia Amorphoides containing 250-270 g/kg of total soluble solids; 60-75 g/kg of triterpene saponins, and a conductivity (5° Brix) of 5.6+/−4; an absorbance at 400 nm (at 1.1% of the product in water, v/v)<0.500 UA; a foam output of (5° Brix, ml)>160 ml; a (direct) pH of 3.9+/−0.3 and a surface tension between 38 and 40 dynes/cm; while the second active component consists of 1,000 cc of a dissolution of granular urea of low Biuret content in pure water, <0.5%, whose content of Ni is about 20%.

8. Use as adjuvant in agriculture of the seedless pod of Gleditsia Amorphoides, as per claim 1, wherein in said fertilizer, the mentioned dissolution is performed in pure water with a pH=7 and lacks the hardness given by the presence of Ca+ and Mg+ ions.

9. Use as adjuvant in agriculture of the seedless pod of Gleditsia Amorphoides, as per claim 1, wherein, for a herbicide of the glyphosate type at 672 g.i.a./ha., 0.06% v/v of the extract is added as adjuvant.

10. Use as adjuvant in agriculture of the seedless pod of Gleditsia Amorphoides, as per claim 1, wherein, in order to formulate herbicides, 1.0 to 1.5% v/v of the extract is added to the final formulation of the product.

11. Use as adjuvant in agriculture of the seedless pod of Gleditsia Amorphoides, as per claim 1, wherein, in an insecticide of the pyrethrum type used to control the leafmining moth (Phyllocnistis Citrella Stainton), between 0.25 and 0.50% of the adjuvant with 0.15% of avemectin is used.

12. Use as adjuvant in agriculture of the seedless pod of Gleditsia Amorphoides, as per claim 1, wherein, for a Paraquat type exfoliating agent, the formulation includes approximately 0.25% v/v of the aqueous extract, an emulsifying oil at 1% and a concentration of 1 l/ha of Paraquat.

13. Use as adjuvant in agriculture of the seedless pod of Gleditsia Amorphoides, as per claim 1, wherein a nitrogenous fertilizer is formulated from 10 to 46 g/100 g of organic Ni and 30 to 60 cc of the aqueous extract, with a pH of 6.9, Biuret of 0.15 g/100 g and surface tension of 36.6 dynes/cm.

14. Use as adjuvant in agriculture of the seedless pod of Gleditsia Amorphoides, as per claim 1, wherein, for the broth of a spraying solution, said aqueous extract is added directly, from 30 to 60 ml per 100 liters of solution.

15. Use as adjuvant in agriculture of the seedless pod of Gleditsia Amorphoides, as per claim 1, wherein extract is added as adjuvant to all types of biocides, such as herbicides, fungicides and insecticides.
